(19) United States
(12) Patent Application Publication
(10) Pub. No.: US 2023/0170202 A1
(43) Pub. Date: Jun. 1, 2023

(54) DETECTING CHEMICAL COMPOUNDS FOR FORENSIC ANALYSIS

(71) Applicant: BOARD OF REGENTS, THE UNIVERSITY OF TEXAS SYSTEM, Austin, TX (US)

(72) Inventors: Livia SCHIAVINATO EBERLIN, Austin, TX (US); Jialing ZHANG, Austin, TX (US); Clara L. FEIDER, Austin, TX (US); Abigail N. GATMAITAN, Austin, TX (US)

(21) Appl. No.: 17/921,756

(22) PCT Filed: Apr. 27, 2021
(86) PCT No.: PCT/US2021/029273

§ 371 Date: Oct. 27, 2022

Related U.S. Application Data
(60) Provisional application No. 63/016,112, filed on Apr. 27, 2020.

(51) Int. Cl. H01J 49/04; G01N 33/487

(52) U.S. Cl. CPC H01J 49/0431; G01N 33/48714; H01J 49/0404; H01J 49/0468

(57) ABSTRACT

In a general aspect, chemical compounds (e.g., drugs, agrochemicals, and explosives) are detected. In some examples, a chemical detection system includes a container, an ionization system, a mass spectrometer, one or more computer systems, a sampling probe, and a control system. The sampling probe is configured to receive a liquid solvent from the container; to hold a fixed volume of the liquid solvent in direct contact with a sample surface for a period of time to form an analyte in the sampling probe; and to deliver the analyte to the ionization system. The ionization system is configured to ionize the analyte. The mass spectrometer is configured to produce mass spectrometry data by processing the ionized analyte provided by the ionization system. The one or more computer systems are configured to analyze the mass spectrometry data to detect a chemical compound present on the sample surface.

### CROSS-REFERENCE TO RELATED APPLICATIONS

This application claims priority to U.S. Provisional Patent Application No. 63/016,112, filed Apr. 27, 2020, entitled “Detecting Chemical Compounds for Forensic Analysis.” The above-referenced priority documents is hereby incorporated by reference in its entirety.

### BACKGROUND

The following description relates to detecting chemical compounds (e.g., drugs, agrochemicals, and explosives) for forensic analysis.

Currently, many technologies that can be employed by law enforcement or regulatory officials lack the sensitivity and specificity needed to accurately identify the compounds present in a substance. Many portable tests, such as the Scott's Reagent test used by law enforcement for the detection of drugs, e.g., cocaine, suffer from high false positive rates and limited sensitivity. Quantitative analysis and identification are typically performed in a laboratory environment using complex equipment and methods. Further, these equipment and methods often require a specialized technician and sample preparation steps that can lead to lengthy analysis time.

### BRIEF DESCRIPTION OF THE DRAWINGS

FIG. 1 is a schematic diagram of an example chemical measurement system.

FIG. 2 is a schematic diagram showing aspects of an example chemical measurement system.

FIG. 3 is a schematic diagram showing aspects of a sampling probe in an example chemical measurement system.

FIG. 4 is a schematic diagram showing aspects of an ionization system in an example chemical measurement system.

FIG. 5 is a flow chart showing an example process for chemical measurement.

FIGS. 6A-6D are example mass spectra of chemical standards including cocaine, oxycodone, hydrocodone, and fentanyl with and without deuterium labeling.

FIGS. 7A-7C are plots of calibration curves of chemical compounds.

FIG. 8 are plots of extracted ion chromatograms of signal intensities as a function of chromatographic retention time.

### DETAILED DESCRIPTION

Quantitative analysis and identification of chemical compounds for forensics applications may require reliable vaporization and ionization to reduce system variability and achieve low limits of detection. In some aspects of what is described here, a chemical measurement system for identification of chemical compounds for forensic applications includes a sampling probe, a control system, an ionization system, and a mass spectrometer. In some examples, the sampling probe is a portable (e.g., handheld) device, and the ionization system receives an analyte from the sampling probe and prepares the analyte for mass spectrometry analysis. The sampling probe may be configured to contact a sample surface (which may contain a chemical compound of interest), to receive a liquid solvent from the control system, to form an analyte by interfacing the liquid solvent with the sample surface (such that the analyte includes at least a portion of the chemical compound from the sample surface mixed with the liquid solvent), and to transfer the analyte to the ionization system. In some instances, the liquid solvent may include an internal standard, e.g., when a quantitative analysis is performed. The analyte received from the sampling probe can be then ionized in the ionization system, and ionization products can be transferred to and processed by the mass spectrometer. Data from the mass spectrometer may then be analyzed to detect (e.g., to detect the presence of, or to detect a level of) the chemical compound of interest on the sample surface.

In some implementations, the methods and systems disclosed here may provide technical advantages. In some implementations, the methods and systems disclosed here can provide robust, reproducible, versatile, and quantitative measurement of a variety of chemical compounds with a wide range of polarities. In some instances, systems with a miniaturized sample probe tip may provide a precise control of the liquid solvent and the analyte (e.g., sub-microliter volume), preventing oversampling or depletion of expensive or limited chemical samples. In some implementations, the methods and systems disclosed here may provide quantitative and robust chemical measurement with high performance, e.g., high accuracy and low limit of detection. Further, equipment may be deployed in a cost-effective manner and provide fast chemical analysis in some cases. In certain implementations, a portable (e.g., handheld) sampling probe and portable design may provide better assistance to law-enforcement officials to perform analysis in the field or at a crime scene. Additionally or alternatively, simplified operational steps and system design may not require experienced professionals to perform such analysis. In some cases, a combination of these and potentially other advantages and improvements may be obtained.

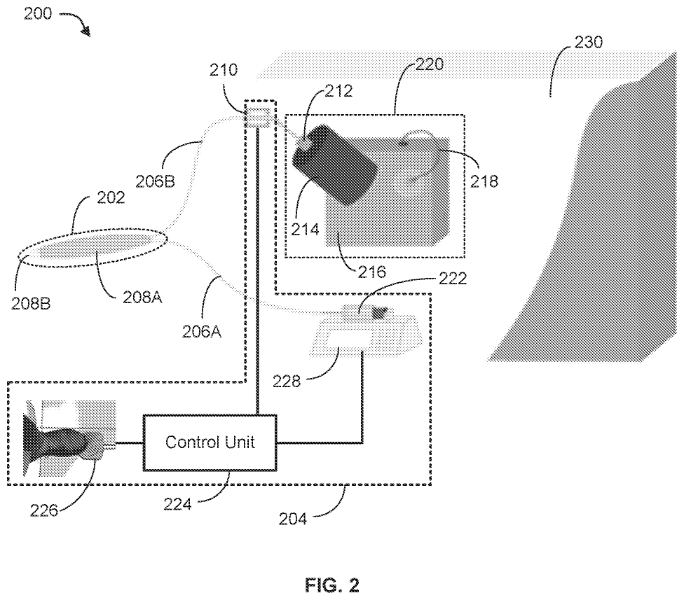

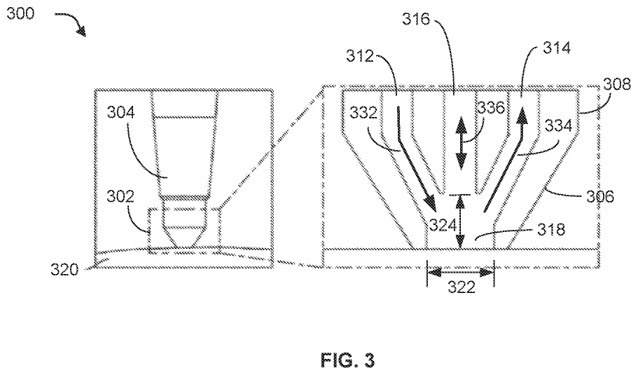

In some implementations, a sampling probe may include a probe tip and a housing. In some implementations, the probe tip, e.g., the probe tip 302 as shown in FIG. 3, includes one mandrel end and one cylindrical end. For example, the mandrel end in a tapered cylindrical shape may be used for contacting the sample surface, and the cylindrical end may be used to engage with a receiving end of the housing. In some implementations, the probe includes three internal channels creating three internal pathways and an internal reservoir. In some examples, the probe tip includes a liquid supply channel (e.g., the liquid supply channel 312), a liquid extraction channel (e.g., the liquid extraction channel 314), and a gas channel (e.g., the gas channel 316). In some implementations, the liquid supply and extraction channels are configured to provide fluidic communication with the control system and the ionization system through respective transfer tubes, e.g., the transfer tubes 206A, 206B as shown in FIG. 2. In some implementations, the sampling probe may be composed of biologically compatible synthetic polymers and may be fabricated using a 3D printing process.

In some implementations, the liquid supply channel is configured for receiving a liquid solvent from an external container, for guiding the liquid solvent to the internal reservoir at the probe tip, where the liquid solvent may be in direct contact with the sample surface, and for filling up at least a portion of the internal reservoir with the liquid solvent. In some implementations, the liquid extraction channel is configured for obtaining an analyte from the internal reservoir by extracting at least a portion of the liquid solvent carrying the chemical compound, and for guiding the analyte to the transfer tube.

In some implementations, the internal reservoir may be in a cylindrical shape which is coupled to the three internal channels of the probe tip. In some implementations, the diameter of the internal reservoir may determine the volume of the liquid solvent exposed to the sample surface and performance aspects of the chemical measurement system, for example spatial resolution, limit of detection, and accuracy.

In some implementations, a fixed volume of liquid solvent is communicated into the probe tip. The fixed volume of fluid can be retained within the internal reservoir while in direct contact with the sample surface for a controlled amount of time, to form an analyte containing molecules from the sample surface. The analyte may then be extracted (e.g., as a single, discrete droplet of fluid) from the internal reservoir through the liquid extraction channel for analysis. In some instances, the analyte is produced by the probe in a non-destructive manner that does not damage the sample surface. For instance, the probe may extract the liquid analyte from a tissue site or tissue sample without causing any detectable damage or destruction to the tissue.

In some implementations, the ionization system may include a transfer capillary, and a discharge needle, which are configured inside of an ionization chamber. In some implementations, the ionization system may be used to ionize both polar and non-polar analytes. In some implementations, the ionization chamber of the ionization system may also contain an inlet tube for collecting an ionized analyte by the mass spectrometer. In some implementation, the transfer capillary, the discharge needle, and the inlet tube are configured in proximity to each other. In some implementations, the transfer capillary is coupled to a transfer tube outside the ionization chamber, e.g. the transfer tube 206B from the sampling probe 202. In some examples, the transfer capillary in the ionization system may be composed of stainless steel and may be heated by a heater to facilitate evaporation of the analyte when existing the transfer capillary. In some implementations, the analyte may be extracted from the internal reservoir of the probe tip through the transfer tube to the transfer capillary by creating a low pressure in the ionization chamber.

In some implementations, the discharge needle may be electrically coupled to a DC source to provide a DC voltage between the discharge needle and a ground. In some examples, when the discharge voltage is applied from the discharge needle to the evaporated analyte from the transfer capillary, the analyte is excited and ionized creating an ionized analyte. In some examples, the ionized analyte containing ionized molecules are transferred out to the mass spectrometer through the inlet tube, where the analyte ions are fragmented into fragment ions which are then filtered and analyzed. In some instances, the ionized molecules are transferred out of the ionization chamber and directly analyzed by the mass spectrometer as intact ionized molecules.

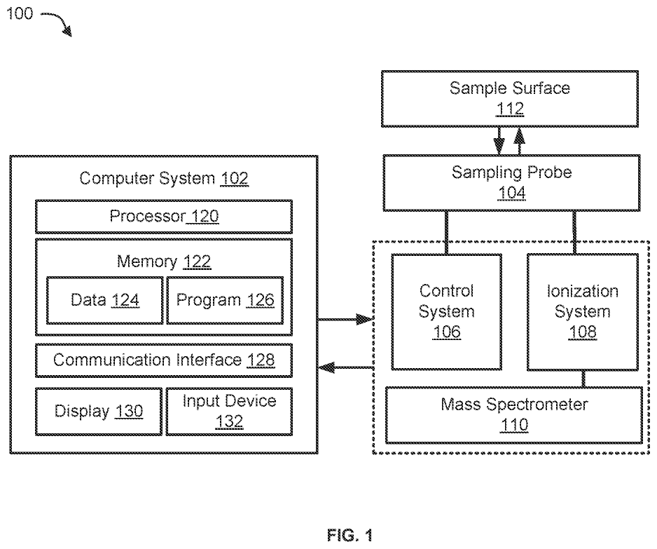

FIG. 1 is a schematic diagram of an example chemical measurement system 100. As shown in FIG. 1, the example system 100 includes a computer system 102, a sampling probe 104, a control system 106, an ionization system 108, and a mass spectrometer 110. In some implementations, the example system 100 may be used for qualitative and quantitative detection and identification of controlled substances, for example drugs, agrochemicals (e.g., pesticides, herbicides, and fungicides), and explosives. In some examples, the chemical measurement system 100 may include additional or different components, and the components may be arranged as shown or in another manner.

In some implementations, the system 100 includes calibration curve data for a large number of compounds (e.g., for many drugs or other types of compounds of interest), and deuterated internal standards in the solvent for each compound. For instance, the system 100 can be calibrated for different compounds both with calibration curves (e.g., stored in memory or accessible through a data network) and the internal standards in the solvent. The system 100 may then be used to analyze any type of object (e.g., a dollar bill or a piece of furniture) to detect the presence and amount of any potential drug or other compound that the system is calibrated for.

In the example shown in FIG. 1, the computer system 102 includes a processor 120, memory 122, a communication interface 128, a display device 130, and an input device 132. In some implementations, the computer system 102 may include additional components, such as, for example, input/output controllers, communication links, power, etc. In some implementations, the computer system 102 may be configured to control operational parameters of and to receive data from the control system 106, the ionization system 108, and the mass spectrometer 110. The computer system 102 can be used to control the ionization system 106 to obtain an analyte by extracting a liquid solvent carrying dissolved chemical compounds. The computer system 102 can be also used to control the ionization system 106 to obtain an ionized analyte for chemical analysis in the mass spectrometer 108. In some implementations, the computer system 102 may be used to implement one or more aspects of the systems and processes described with respect to FIGS. 2, 3, 4, and 5, or to perform other types of operations. In some implementations, the computer system 102 includes a separate control unit associated with and providing specific control functions to the control system 106.

In some implementations, the computer system 102 may include a single computing device, or multiple computers that operate in proximity to the rest of the example system 100 (e.g., the control system 106, the ionization system 108 and the mass spectrometer 110). In some implementations, the computer system 102 may communicate with the rest of the example system 100 via the communication interface 128 through a communication network, e.g., a local area network (LAN), a wide area network (WAN), an inter-network (e.g., the Internet), a network comprising a satellite link, and peer-to-peer networks (e.g., ad hoc peer-to-peer networks).

In some implementations, the sampling probe 104 may be configured to provide fluidic communication with the control system 106 and the ionization system 108 via transfer tubes. In some aspects of operation, the sampling probe 104 receives liquid solvent from the control system 106, guides the liquid solvent to a sample surface 112 with chemical compounds of interest, obtains an analyte by extracting at least a portion of the liquid solvent with dissolved chemical compounds from the sample surface 112, and guides the analyte to the ionization system 108. In some implementations, the sample surface 112 is a surface of a forensic object of interest, a surface of paper money, a biologic tissue, or another object. The biological tissue can be, for example, an in-vivo tissue site, an ex-vivo tissue sample, or another type of biological tissue. In some implementations, the sampling probe 104 may include a probe tip, which may include multiple internal liquid/gas channels and an internal reservoir, e.g., the channels 312, 314, 316 and the internal reservoir 318 as shown in FIG. 3 or in another manner. In some implementations, the sampling probe 104 may be composed of materials, such as synthetic polymers that are biologically compatible and resistant to chemical compounds under measurement. In some examples, the sampling probe 104 may be implemented as the sampling probes 202, 300 as shown in FIGS. 2-3 or in another manner.

The example control system 106 controls the movement of fluid in the system 100. In some implementations, the control system 106 includes a mechanical pumping system and one or more mechanical valves. In some instances, the mechanical pumping system includes a mechanical pump that is controlled by the computer system 102, which may provide high-precision, microfluidic dispensation of the liquid solvent to the internal reservoir of the sampling probe 104. In some implementations, the liquid solvent may be polar or non-polar, which may include sterile water, a type of alcohol, an internal standard, or a combination. In some implementations, the control system 106 may be implemented as the control system 204 as shown in FIG. 2 or in another manner. In some instances, a control unit of the control system 106 (e.g., the control unit 224) may be configured to trigger and control a sampling process by controlling the mechanical pumping system and the one or more mechanical valves. Simultaneously, the control unit of the control system 106 may be configured to trigger a data collection process by the mass spectrometer 110.

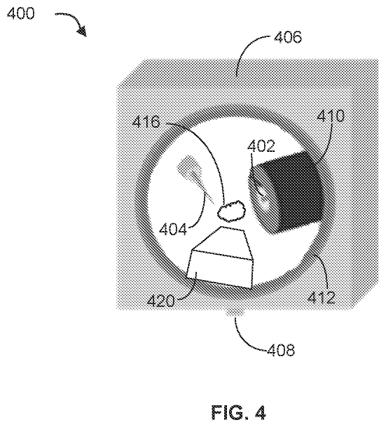

In some implementations, the ionization system 108 includes a transfer capillary, a discharge needle, and an inlet tube, which may be enclosed in an ionization chamber. In some examples, the ionization chamber is coupled to a vacuum pump. In some implementations, the transfer capillary is coupled to a transfer tube outside of the ionization chamber to receive the analyte from the sampling probe 104. In some implementations, a low pressure created in the ionization chamber by the vacuum pump may facilitate extraction of the analyte from the sampling probe 104, to the transfer capillary and eventually to the ionization chamber. In some instances, the transfer capillary is heated by a heater so that the analyte may be evaporated when exiting the transfer capillary. In some implementations, the low pressure in the ionization chamber may be in a range of 100 to 1000 mbar, 500 to 1000 mbar, or in another range. In some examples, the pressure in the ionization chamber is about 600 mbar. In some implementations, the discharge needle is used to apply a discharge voltage (e.g., up to 8 kV) to the analyte aspirated from the transfer capillary to obtain an ionized analyte. In some implementations, the ionized analyte contains a gas cluster of ionization products of the liquid solvent and dissolved chemical compounds. In some implementations, the ionization system 108 may be implemented as the ionization system 400 as shown in FIG. 4 or in different manner.

In some implementations, the ionized analyte is collected by and transferred through the inlet tube to the mass spectrometer 110. In some implementations, the mass spectrometer 110 may include a mass selector and a mass analyzer, which are configured to separate and identify the ionization products in the ionized analyte according to their mass-to-charge (m/z) ratio. In some implementations, the mass spectrometer 110 may output a set of mass spectra (e.g., intensity of ionization products vs. m/z ratio plot) to the computer system 102, which may be stored in the memory 122, analyzed by running a program 126 and results may be further displayed on the display 130. In some implementations, the mass spectrometer 110 may be implemented as the mass spectrometer 230 as shown in FIG. 2 or in different manner.

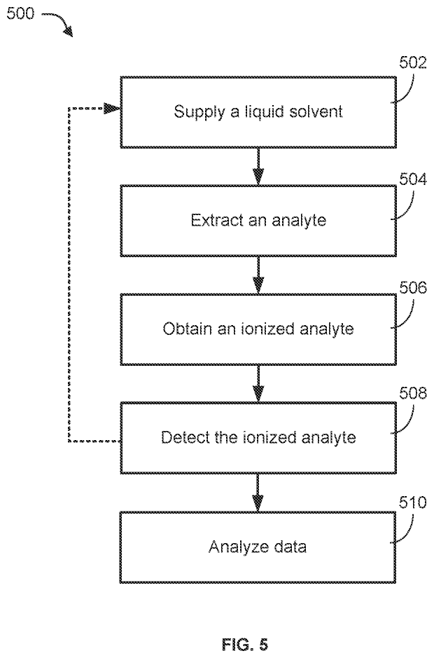

In some implementations, some of the processes and logic flows described in this specification can be automatically performed by one or more programmable processors, e.g. processor 120, executing one or more computer programs to perform actions by operating on input data and generating output. For example, the processor 120 can run the programs 126 by executing or interpreting scripts, functions, executables, or other modules contained in the programs 126. In some implementations, the processor 120 may perform one or more of the operations described, for example, with respect to FIG. 5.

In some implementations, the processor 120 can include various kinds of apparatus, devices, and machines for processing data, including, by way of example, a programmable data processor, a system on a chip, or multiple ones, or combinations, of the foregoing. In certain instances, the processor 120 may include special purpose logic circuitry, e.g., an Arduino board, an FPGA (field programmable gate array), an ASIC (application specific integrated circuit), or a Graphics Processing Unit (GPU) for running the deep learning algorithms. In some instances, the processor 120 may include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. In some examples, the processor 120 may include, by way of example, both general and special purpose microprocessors, and processors of any kind of digital computer.

In some implementations, the processor 120 may include both general and special purpose microprocessors, and processors of any kind of quantum or classic computer. Generally, a processor 120 receives instructions and data from a read-only memory or a random-access memory or both, e.g. memory 122. In some implementations, the memory 122 may include all forms of non-volatile memory, media, and memory devices, including by way of example semiconductor memory devices (e.g., EPROM, EEPROM, flash memory devices, and others), magnetic disks (e.g., internal hard disks, removable disks, and others), magneto optical disks, and CD ROM and DVD-ROM disks. In some cases, the processor 120 and the memory 122 can be supplemented by, or incorporated in, special purpose logic circuitry.

In some implementations, the data 124 stored in the memory 122 may include, operational parameters, a standard reference database and output data. In some implementations, the standard reference database may include a mass spectral reference library, which may be used for identification of unknown chemical compounds. In some implementations, the reference database may further include calibration curves (e.g., intensity vs. concentration) for quantitative analysis of chemical compounds. In some implementations, the programs 126 can include software applications, scripts, programs, functions, executables, or other modules that are interpreted or executed by the processor 120. In some implementations, the programs 126 may include machine-readable instructions for performing deep learning algorithms. In some instances, the programs 126 may include machine-readable instructions for delivering the liquid solvent to the sampling probe, collecting the analyte from the sampling probe, obtaining the ionized analyte for mass spectral analysis. In some instances, the programs 126 may obtain input data from the memory 122, from another local source, or from one or more remote sources (e.g., via a communication link). In some instances, the programs 126 may generate output data and store the output data in the memory 122, in another local medium, or in one or more remote devices (e.g., by sending the output data via the communication network 106). In some examples, the programs 126 (also known as, software, software applications, scripts, or codes) can be written in any form of programming language, including compiled or interpreted languages, declarative or procedural languages. In some implementations, the programs 126 can be deployed to be executed on the computer system 102.

In some implementations, the communication interface 128 may be connected to a communication network, which may include any type of communication channel, connector, data communication network, or other link. In some instances, the communication interface 128 may provide communication with other systems or devices. In some instances, the communication interface 128 may include a wireless communication interface that provides wireless communication under various wireless protocols, such as, for example, Bluetooth, Wi-Fi, Near Field Communication (NFC), GSM voice calls, SMS, EMS, or MMS messaging, wireless standards (e.g., CDMA, TDMA, PDC, WCDMA, CDMA2000, GPRS) among others. In some examples, such communication may occur, for example, through a radio-frequency transceiver or another type of component. In some instances, the communication interface 128 may include a wired communication interface (e.g., USB, Ethernet) that can be connected to one or more input/output devices, such as, for example, a keyboard, a pointing device, a scanner, or a networking device such as a switch or router, for example, through a network adapter.

In some implementations, the communication interface 128 can be coupled to input devices and output devices (e.g., the display device 130, the input device 132, or other devices) and to one or more communication links. In the example shown, the display device 130 is a computer monitor for displaying information to the user or another type of display device. In some implementations, the input device 132 is a keyboard, a pointing device (e.g., a mouse, a trackball, a tablet, and a touch sensitive screen), or another type of input device, by which the user can provide input to the computer system 102. In some examples, the computer system 102 may include other types of input devices, output devices, or both (e.g., mouse, touchpad, touchscreen, microphone, motion sensors, etc.). The input devices and output devices can receive and transmit data in analog or digital form over communication links such as a wired link (e.g., USB, etc.), a wireless link (e.g., Bluetooth, NFC, infrared, radio frequency, or others), or another type of link.

In some implementations, other kinds of devices may be provided for interaction with a user as well; for example, feedback provided to the user can be any form of sensory feedback, e.g., visual feedback, auditory feedback, or tactile feedback; and input from the user can be received in any form, including acoustic, speech, or tactile input. For example, the sampling probe 104 may contain a control element (e.g., button, pedal, etc.) which may be used as a controller to initiate, interrupt, restart, or terminate a detection process (e.g., the pedal 226 as shown in FIG. 2). In some instances, a graphic user interface (GUI) may be used to provide interactions between a user and the chemical measurement system 100. In certain instances, the GUI may be communicably coupled to the computer system 102. For example, when the control system 106 is activated (e.g., by pushing on the pedal 226 in FIG. 2), the GUI can simultaneously initiate an ionization process on the ionization system 108 and a chemical analysis process on the mass spectrometer 110. For example, when the chemical analysis process is completed, the GUI can output and display a report with analysis results.

FIG. 2 is a schematic diagram of a portion of a chemical measurement system 200. In the example shown in FIG. 2, the chemical measurement system 200 includes a sampling probe 202, a control system 204, an ionization system 220 and a mass spectrometer 230. As shown in FIG. 2, the sampling probe 202 is coupled between the control system 204 and the ionization system 220 through transfer tubes 206A, 206B. In some examples, the chemical measurement system 200 may include additional or different components, and the components may be arranged as shown or in another manner.

In the example shown in FIG. 2, the sampling probe 202 includes a housing 208A and a probe tip 208B. In some implementations, the housing 208A may provide a grip for being used as a hand-held sampling probe. In some implementations, the housing 208A may include a control element, e.g., a trigger or button. For example, the control element may be used to control the liquid solvent transferring through the sampling probe 202. In some instances, the control element may be separated from the housing 208A, e.g., configured as a foot pedal. For another example, the control element may be coupled to a mechanism which may be used to eject the probe tip 208B. In some implementations, the sampling probe 202 may be composed of materials, such as synthetic polymers that are biologically compatible and resistant to chemical compounds under measurement. For example, the materials for the sampling probe 202 may be compatible with a variety of liquid solvent (e.g., polar, or non-polar) that is used for extracting and carrying an analyte to the ionization system 220. In some examples, the synthetic polymers that may be used for fabricating the sampling probe 202 may include Polydimethylsiloxane (PDMS), or Polytetrafluoroethylene (PTFE). In some implementations, the probe tip 208B may use the same material as the housing 208A, different materials or different compositions.

In some implementations, the sampling probe 202 may be manufactured using a 3D printing process, a machining process, or another process. In some implementations, the housing 208A of the sampling probe 202 may include two internal channels which are fluidically coupled with respective transfer tubes 206A, 206B and respective channels in the probe tip 208B. In some implementations, the transfer tubes 206A, 206B may be configured for supplying a liquid solvent from the control system 204 to the probe tip 208B and for transferring an analyte which includes at least a portion of the liquid solvent with dissolved chemical compounds from the probe tip 208B to the ionization system 220. The sampling probe 202 may also include a gas channel (e.g., an open port that receives air from the surrounding atmosphere) that allows liquid to be flushed from the sampling probe 202, for example, between uses or at other instances.

In some implementations, the probe tip 208B may be detachable from the housing 208A, which can be disposed and replaced if contaminated, e.g., after a certain number (e.g., one or more) of regular uses or when switching between different samples. In some cases, the probe tip 208B may include internal channels that are fluidically coupled to the respective channels in the housing 208A and further to the respective transfer tubes 206A, 206B. In some implementations, the probe tip 208B may be integrated with the housing 208A as a monolithic structure. In some implementations, the probe tip 208B may be implemented as the probe tip 302 as shown in FIG. 3 or in another manner.

In some implementations, the control system 204 may include a solvent container and a mechanical pumping system 228. In some instances, the mechanical pumping system 228 may contain one or more mechanical pumps. In some instances, the one or more mechanical pumps may be programable. In certain examples, the one or more mechanical pumps may be controlled by a computer system, e.g., the computer system 102 in FIG. 1. In some implementations, a mechanical pump may include a syringe pump, a peristatic pump, or another type of pump, which can provide high-precision, microfluidic dispensation of the liquid solvent 222 from the solvent container to the probe tip 208B, e.g., the internal reservoir 318 of the probe tip 302 as shown in FIG. 3. In some implementations, each of the one or more mechanical pumps may be equipped with separate solvent containers containing different types of liquid solvents. In some instances, different types of liquid solvents may be selected or mixed according to the types of chemical compounds and initial measurement results. In the example shown in FIG. 2, the liquid solvent 222 in a container (e.g., syringe) is delivered to the sampling probe 202 through a first transfer tube 206A. In some implementations, the control system 204 can supply a controlled volume of liquid solvent to the sampling probe 202 at a controlled flow rate according to the design of the probe tip 208B, e.g., the length of the first transfer tube 206A, the volume of the internal reservoir 318 and the diameter of the liquid channels 312, 314 as shown in FIG. 3.

As shown in FIG. 2, the control system 204 further includes one or more valves on the transfer tubes 206A, 206B. In some implementations, each of the one or more valves is configured to control a fluidic flow (e.g., start or stop a fluidic flow) in respective transfer tubes. In some implementations, each of the one or more valves may be mechanically activated and electrically controlled by a computer system, e.g., the computer system 102 as shown in FIG. 1. In some examples, the one or more valves 210 may include a pinch valve, a squeeze valve, other type of valve, or a combination. In some instances, the valve 210 on a second transfer tube 206B is a high-speed actuated pinch valve for controlling aspiration and extraction of the analyte to the ionization system 220. In some instances, the control system 204 is communicably coupled with a control unit 224. In some instances, the control unit 224 may include an Arduino board to control motions of the mechanical pumping system 228 and the one or more valves 210. As shown, the control unit 224 can be activated by pushing a pedal 226 and deactivated by releasing the pedal 226. In some instances, when activated, the control unit 224 may also initiate a data collection process performed by the mass spectrometer 230.

In some implementations, the liquid solvent 222 may be polar or non-polar. For example, the liquid solvent 222 in the example control system 204 includes sterile water. In some instances, the liquid solvent 222 further includes a type of alcohol, e.g., ethanol, methanol, and a combination. In some examples, the liquid solvent 222 includes acetonitrile. In certain examples, the liquid solvent 222 may contain acid, for example acetic acid, hydrochloric acid, or another type of acid. In some implementations, the liquid solvent 222 contains a deuterated chemical compound as an internal standard.

In some implementations, the transfer tubes 206A, 206B may have a length in the range of approximately half a meter to one or more meters (e.g., a length in the range of approximately 0.5 m to 1.5 m, or in another range) to allow free handheld use of the sampling probe 202 by an operator without geometrical or spatial constraints.

In some implementations, the ionization system 220 includes a transfer capillary 212, and a discharge needle 218 configured inside of an ionization chamber 216. In some aspects of operation, the ionization system 220 receives and ionizes an analyte. In some implementations, the transfer capillary 212 is coupled to the second transfer tube 206B and is further enclosed by a heater 214. In some implementations, the heater 214 may be a ceramic heater which is configured to heat the transfer capillary 212 to a temperature in a range of 200-400 degree Celsius. In some implementations, the ionization chamber 216 may be coupled to a vacuum pump (not shown) to control a pressure in the ionization chamber 216. In certain instances, the pressure may be used to facilitate the extraction of the analyte from the probe tip 208B to the transfer capillary 212 and further into the ionization chamber 216. In some implementations, the ionization system 220 may be implemented as the ionization system 400 as shown in FIG. 4 or in another manner. In some implementations, when a discharge voltage is applied on the discharge needle 218 to the analyte aspirated from the transfer capillary 212, the analyte is excited and ionized creating an ionized analyte, e.g., a gas cluster of ionization products. In some examples, the ionized analyte includes ionized molecules from the liquid solvent and the dissolved chemical compound. In some implementations, the ionized analyte may be transfer out to the mass spectrometer 230, e.g., through an inlet tube located in proximity to the discharge needle 218. In some aspects of operation, the ionization products in the ionized analyte are filtered, captured, and analyzed by the mass spectrometer 230. In some implementations, prior to reaching the mass spectrometer, the ionized analyte may be collected and delivered to an ion optic system. In some instances, the ion optic system may be configured to filter neutral species in the ionized analyte, to allow ions passing through, and to eliminate contamination of the mass spectrometer.

In some implementations, the mass spectrometer 230 may include a mass selector and a mass analyzer. In some implementations, the mass selector may separate the analyte ions (e.g., intact ions) according to their mass-to-charge (m/z) ratio based on dynamics of charged particles in electric and magnetic field in vacuum. The mass selector may include a set of magnets providing a magnetic field, which the analyte ions travel through. In some instances, the mass selector may use the magnetic field to alter the path of the analyte ions so that they can be separated according to their charges and masses. The mass analyzer may include detectors to identify ions. In some examples, the mass spectrometer 230 can output a set of mass spectra (or mass spectrometry data in another format) for data analysis. In some instances, the analyte ions may be further fragmented by an internal fragmentation source which produces fragment ions which can be filtered by the mass selector and analyzed by the mass analyzer.

FIG. 3 is a schematic diagram showing aspects of a sampling probe 300 in an example chemical measurement system. As shown in FIG. 3, the sampling probe 300 includes a probe tip 302 and a housing 304. The example probe tip 302 includes one mandrel end 306 in a tapered cylindrical shape which is used for contacting a sample surface 320, and one cylindrical end 308 which is used to engage with a receiving end of the housing 304. In some implementations, the cylindrical end 308 makes an air-tight seal with the receiving end of the housing 304. In some examples, the probe tip 302 may include additional or different components, and the components may be arranged as shown or in another manner.

As shown in a cross-sectional view of the probe tip 302 in FIG. 3, the probe tip 302 includes three distinct internal channels, including a liquid supply channel 312, a liquid extraction channel 314, and a gas channel 316. In some implementations, the three internal channels 312, 314, 316 are aligned with respective internal channels (not shown) in the receiving end of the housing 304 to provide fluidic communication with transfer tubes. In some instances, the transfer tubes may be implemented as the transfer tubes 206A, 206B as shown in FIG. 2 or in another manner. In some implementations, the three internal channels 312, 314, 316 may be directly coupled with transfer tubes that extends through the housing 304 from the end opposite to the receiving end to the receiving end of the housing 304 or may be coupled with the transfer tubes in other manner to allow liquid and gas flow.

In some implementations, the housing 304 is configured to provide fluidic communication with a control system and an ionization system through respective transfer tubes, e.g., the transfer tubes 206A, 206B as shown in FIG. 2. In some implementations, the housing 304 and the probe tip 302 may be composed of biologically compatible synthetic polymers. In some implementations, the housing 304 and the probe tip 302 may be fabricated using a 3D printing process, a machining, or another type of fabrication process.

In some implementations, the sample surface 320 may be a surface of a solid substrate. The sample surface 320 may be a surface of a forensic object of interest, a surface of paper money, a biologic tissue, or another object. The biological tissue can be, for example, an in-vivo tissue site, an ex-vivo tissue sample, or another type of biological tissue. In some examples, the sample surface 320 may be hydrophobic to prevent spreading of a liquid solution on the sample surface. For example, the sample surface 320 can be a PTFE-coated glass slide. In some implementations, the sample surface 320 may make a liquid-tight seal with the mandrel end 306 of the probe tip 302 to prevent leakage of the liquid solvent from the internal reservoir 318. In some implementations, the sample surface 320 may contain at least one chemical compound of interest. In some examples, a controlled amount of a chemical compound may be deposited on a sample surface by dropping a fixed volume of a calibration solution on the sample surface followed by drying in air at room temperature. In some instances, the calibration solution contains a known concentration of the chemical compound dissolved in a stock solution. In some instances, calibration solutions at lower concentrations are further prepared by performing a series of dilutions to the calibration solution at a higher concentration.

In some aspects of operation, the liquid supply channel 312 receives the liquid solvent from an external container, guides the liquid solvent to the internal reservoir 318 at the probe tip 302, where the liquid solvent may be in direct contact with the sample surface 320, and fills at least a portion of the internal reservoir 318 with the liquid solvent. The liquid supply channel 312 may provide a first internal pathway 332 in the probe tip. In some implementations, the liquid solvent may be received from the external container as a part of a control system, e.g., the control system 204 as shown in FIG. 2 or another type of control system.

In some implementations, the internal reservoir 318 may have a cylindrical shape and be coupled to the liquid supply channel 312. In certain examples, the liquid solvent received from the liquid supply channel 312 in the internal reservoir 318 makes direct contact with the sample surface 320 and at least a portion of the chemical compounds on the sample surface 320 is mixed (e.g., dissolved) into the liquid solvent. In some implementations, the diameter 322 of the internal reservoir 318 is determined by, for example, the size of the sample surface 320 and the amount of chemical compounds on the sample surface 320. In some instances, the diameter 322 and height 324 of the internal reservoir 318 may determine the volume of the liquid solvent exposed to the sample surface 320 and performance aspects of the chemical measurement system, for example a spatial resolution, limit of detection, and accuracy. In some instances, the diameter of the internal reservoir 318 of the probe tip 302 may be in a range of 1.5-5.0 mm. For example, when the diameter 322 of the internal reservoir is 2.77 mm and the height 324 of the internal reservoir 318 is 1.7 mm, the volume of a liquid solvent that is contained in the internal reservoir 318 is 10 microliter (μL). For another example, when the diameter of the internal reservoir 318 is 1.5 mm and the height 324 is 2.5 mm, the volume of a liquid solvent that is contained in the internal reservoir 318 is 4.4 μL. The internal reservoir 318 may have a different shape, aspect ratio, size, or dimension.

In some instances, the liquid extraction channel 314 provides a second, distinct internal pathway 334 in the probe tip 302. In some aspects of operation, the liquid extraction channel 314 obtains an analyte by extracting at least a portion of the liquid solvent carrying chemical compounds from the internal reservoir 318, and guides the analyte to the transfer tube that is coupled to an ionization system. In some implementations, the analyte from the internal reservoir 318 may be extracted by a vacuum pump coupled to the ionization system (e.g., the ionization chamber 406 of the ionization system 400 as shown in FIG. 4). In some implementations, a low pressure created on one end of the transfer tube may facilitate liquid aspiration to drive the analyte from the internal reservoir 318 to the ionization system through the liquid extraction channel 314.

In some implementations, the gas channel 316 provides a third, distinct internal pathway 336 in the probe tip 302. In some instances, the gas channel 316 is configured for preventing collapse of the sampling probe, transfer tubes and the control system during the extraction. In some instances, the gas channel 316 is open to atmosphere (e.g., air). In some instances, diameters of the liquid supply channel 312, the liquid extraction channel 314 and the gas channel 316 may be equal to 0.8 mm. Gas from the gas channel 316 may be used to push the liquid out of the liquid extraction channel 314 to the ionization chamber.

FIG. 4 is a schematic diagram of a portion of an ionization system 400 in a chemical measurement system. As shown in FIG. 4, the ionization system 400 includes a transfer capillary 402, a discharge needle 404, an inlet tube 420, which are configured in an ionization chamber 406. In some implementations, the ionization system 400 may be used to ionize both polar and non-polar molecules in liquid solvent or chemical compounds dissolved. In some implementations, the ionization system 400 operates in a positive or negative ion mode, where positively or negatively charged molecular ions are generated and subsequently captured and detected by the mass spectrometer. In some examples, the ionization system 400 may include additional or different components, and the components may be arranged as shown or in another manner.

In some implementations, the transfer capillary 402 is configured along a direction perpendicular to the discharge needle 404. In some examples, a distance between tips of the discharge needle 404 and the transfer capillary 402 is about 3 cm or another distance. In some implementations, the transfer capillary 402 is a stainless-steel tube, which may be coupled to a transfer tube, e.g. the transfer tube 206B from the sampling probe 202. In the example shown in FIG. 4, the transfer capillary 402 is enclosed by a ceramic heater 410. In some instances, the transfer capillary 402 may be heated by the heater 410 to a temperature in a range of 200-400 degree Celsius or another temperature range. In some implementations, the tip of the transfer capillary 402 is flush with the end of the ceramic heater 410 to allow a thorough removal of the analyte from the transfer capillary 402 and reduce residual left in the ceramic heater 410. In some implementations, aspiration of the analyte from the transfer capillary 402 may be facilitated by a vacuum pump coupled to a vacuum port 408 on the ionization chamber 406. In some implementations, the ionization chamber 406 may be composed of metal, e.g., aluminum (Al).

In some implementations, the discharge needle 404 may be electrically coupled to a DC source. In some instances, the DC source provides a DC voltage between the discharge needle 402 and a ground e.g., the ionization chamber wall. In some examples, the DC voltage (e.g., up to 8 kV or another voltage) may be applied on the discharge needle 404 for ionization of the analyte aspirated from the transfer capillary 402. In some examples, a constant DC voltage of 4-6 kV is applied during the time of the aspirat of the analyte. In some implementations, the DC source may sustain a current in a range of 2-6 μA or another range. In some examples, the discharge needle 404 is a corona discharge needle creating corona discharges.

In some implementations, when the discharge voltage is applied from the discharge needle 404 to the analyte aspirated from the tip of the transfer capillary 402, the analyte containing the liquid solvent and dissolved chemical compounds are excited and ionized creating an ionized analyte 416, e.g., a gas cluster of ionization products. In some examples, the ionized analyte 416 includes ionized molecules from the solvent and the chemical compound collected from the sample surface.

In some implementations, the inlet tube 420 is used for collecting and delivering the ionized analyte to a mass spectrometer. In some implementations, the transfer capillary 402, the discharge needle 404 and the inlet tube 420 may be configured in proximity to each other. In the example shown in FIG. 4, the transfer capillary 402, the discharge needle 404 and the inlet tube 420 are closely arranged and triangularly aligned. In some implementations, the ionized analyte 416 are transferred out to a mass spectrometer through the inlet tube 420 by creating a low pressure in the inlet tube 420. In some examples, a first pressure in the inlet tube is lower than a second pressure in the ionization chamber. In some implementations, the ionized molecules in the ionized analyte 416 are captured and analyzed by a mass analyzer in the mass spectrometer, e.g., the mass spectrometer 136, 220 as shown in FIGS. 1-2. In some implementations, prior to the mass spectrometer, the ionized analyte 416 may be collected and delivered to an ion optic system. In some instances, the ion optic system may be configured to filter neutral species in the ionized analyte 416 and allow ions passing through. In some instances, the ion optic system may be used to eliminate contamination of the mass spectrometer.

FIG. 5 is a flow chart showing an example process 500 for chemical measurement. In some implementations, the example process 500 may be an automated process used for identification of unknown chemical compounds. The example process 500 may be used for qualitative and quantitative identification and detection of controlled substances, including drugs, agrochemicals, and explosives. The example process 500 may be performed, for example, by a chemical measurement system. For instance, operations in the example process 500 may be performed by the chemical measurement system shown in FIGS. 1-4 or another type of chemical measurement system with additional or different components. The example process 500 may include additional or different operations, including operations performed by additional or different components, and the operations may be performed in the order shown or in another order. In some cases, operations in the example process 500 can be combined, iterated, or otherwise repeated or performed in another manner.

At 502, a liquid solvent is supplied. In some implementations, a first portion of the liquid solvent may be supplied to a sample surface to extract (e.g., dissolve or otherwise mix with) a chemical compound of interest. In some implementations, the sample surface may be implemented as the sample surface 320 as shown in FIG. 3. In some examples, the chemical compound may be deposited on the sample surface by dropping a fixed volume of a solution with a known concentration of the chemical compound on the sample surface followed by drying in air at room temperature.

In some implementations, the first portion of the liquid solvent is supplied to the sample surface with controlled volume and flow rate. In some implementations, a control system (e.g., the control system 204 as shown in FIG. 2) may be used to supply the liquid solvent to a sampling probe with an internal reservoir at an opening (e.g., the sampling probe 300 as shown in FIG. 3), where the first portion of the liquid solvent may be in direct contact with the sample surface. In some implementations, the control system may be controlled by a computer system (e.g., the computer system 102 as shown in FIG. 1). In certain examples, the first portion of the liquid solvent may be delivered to the internal reservoir of the sampling probe for a first time period. For example, a syringe pump may take 2 seconds to deliver 10 μL of the liquid solvent to the internal reservoir ata flow rate of 300 μL/min.

In some implementations, the first portion of the liquid solvent after being delivered to the internal reservoir of the sampling probe may interact with at least a portion of the chemical compound at the sample surface causing the at least a portion of the chemical compound to dissolve in (or otherwise mix with) the first portion of the liquid solvent contained in the internal reservoir. In some implementations, the first portion of the liquid solvent contains a fixed volume. In certain instances, the first portion of the liquid solvent with the chemical compound forms a part of an analyte for mass spectral analysis. In some implementations, the first portion of the liquid solvent may be allowed to interact with the chemical compound on the sample surface for a second time period. In some implementations, the second time period may be determined by one or more of the following, a type of solvent (e.g., polar, or non-polar), temperature, solubility of the chemical compounds in a solvent, and morphology of the chemical compounds on the sample surface. In some instances, the second time period is 3 seconds, or another duration of time may be used.

At 504, an analyte is extracted. The fixed volume of the fluid can be retained within the internal reservoir to form an analyte. In some implementations, the analyte may include the first portion of the liquid solvent mixed with the chemical compound. In some implementations, extraction of the analyte from the sample surface may be performed by applying a first pressure on one end of a transfer tube coupled to the sampling probe. In some instances, the first pressure may be lower than the atmospheric pressure. In some implementations, the analyte may be extracted into an ionization system (e.g., the ionization system 220 as shown in FIG. 2). When the analyte is extracted from the internal reservoir, the analyte is extracted as a single, discrete droplet of fluid. In some examples, after the analyte is extracted from the sampling probe, the sampling probe (e.g., at least part of the internal reservoir, liquid extraction channel 314 and the transfer tube 206B) may be flushed with a second portion of the liquid solvent. In some instances, the second portion of the liquid solvent may be not in contact with the sample surface. In certain instances, the sampling probe may be removed from the sample surface when the sampling probe is flushed with the second portion of the liquid solvent. In certain examples, the second portion of the liquid solvent used for flushing at least part of the internal reservoir and transfer tubes may be also extracted as a part of the analyte into the ionization system. In some implementations, the analyte may be heated during the extraction and evaporated. For example, the analyte is extracted via the transfer capillary 402 in the ionization chamber 406 of the ionization system 400 as shown in FIG. 4 or in another manner. In some implementations, the extraction of the analyte (e.g., the first and second portions of the liquid solvent) can be performed for a third time period which may be in a range of 10-15 seconds or another time duration.

At 506, an ionized analyte is obtained. In some implementations, the analyte extracted and aspirated into the ionization system may be ionized at the same time during the third time period. In some examples, the ionization process may be operated under the first pressure. For example, the first pressure may be in a range of 100-1000 mbar, e.g., 600 mbar, or another pressure. In some implementations, the ionized analyte may be obtained using a discharge needle, e.g., the ionization system 400 as shown in FIG. 4 or in another manner. In some implementations, the ionized analyte may contain a gas cluster of ionization products of the liquid solvent and the dissolved chemical compound.

At 508, the ionized analyte is processed. In some implementations, the ionized analyte may be collected by a mass spectrometer. In some implementations, when traveling in the mass spectrometer, the ionization products in the ionized analyte may be separated and identified according to their mass-to-charge (m/z) ratio. In some examples, the ionized analyte may be scanned one or more times during the third time period. In some implementations, the mass spectrometer 110 may be implemented as the mass spectrometer 230 as shown in FIG. 2 or in different manner.

At 510, data is analyzed. In some implementations, a set of mass spectra collected by the mass spectrometer may be output to the computer system, which may be further stored and analyzed. In some implementations, a molecular profile of the analyte may be obtained by averaging multiple scans (e.g., the set of mass spectra) collected in the third time period by the mass spectrometer. In certain instances, the molecular profile may be further compared or fitted to standard reference spectra in a mass spectral reference library in order to identify composition of the chemical compound. In some instances, a mass spectral background from a blank liquid solvent may be subtracted or filtered. In some implementations, molecular features in the molecular profile (e.g., peaks in the mass spectra) may be selected and intensities of the molecular features may be compared to one or more calibration curves previously obtained to quantitatively determine a composition of the chemical compound.

In some implementations, the mass spectrometer may be a tandem mass spectrometer, where the cluster of fragment ions created by the internal fragmentation source is further filtered by selecting one or more particular fragment ions from the cluster according to m/z ratios. In some examples, the one or more particular fragment ions have the most intense peak in a mass spectrum. In certain examples, the one or more particular fragment ions do not have the most intense peak in the mass spectrum. In some examples, the tandem mass spectrometer may filter and select the one or more particular fragment ions within a few milliseconds. In some instances, intensities of a particular fragment ion obtained during multiple scans using the tandem mass spectrometer may be then averaged. In some examples, a calibration curve may be obtained by recursively performing the example process 500 on sample surfaces prepared using different concentrations of solutions. In some implementations, an extracted-ion chromatogram (EIC) may be obtained by reorganizing the mass spectra to separate one or more particular fragment ions of interest according to the m/z ratios. In certain examples, the EIC may be performed on one or more particular fragment ions with particular m/z ratios. In some implementations, the intensity value used to determine the calibration curve may be obtained from the EIC.

In some implementations, a total detection time at one location of the sample surface, e.g., a summation of the first, second, and third time period, may be in a range of 15-25 seconds. In some implementations, operations 502-508 in the process 500 may be completed in 10 seconds or less. In some implementations, the process may be automatically operated by the computer system. In some implementations, operations 502-508 may be performed in a recursive manner to obtain replicate measurement at different locations on the sample surface with chemical compounds prepared using solutions with the same concentration. In some implementations, between each replicate measurement or when switching between sampling on different sample surfaces, a cleaning process to wash the transfer tube and the sampling probe can be separately performed to minimize cross-contamination.

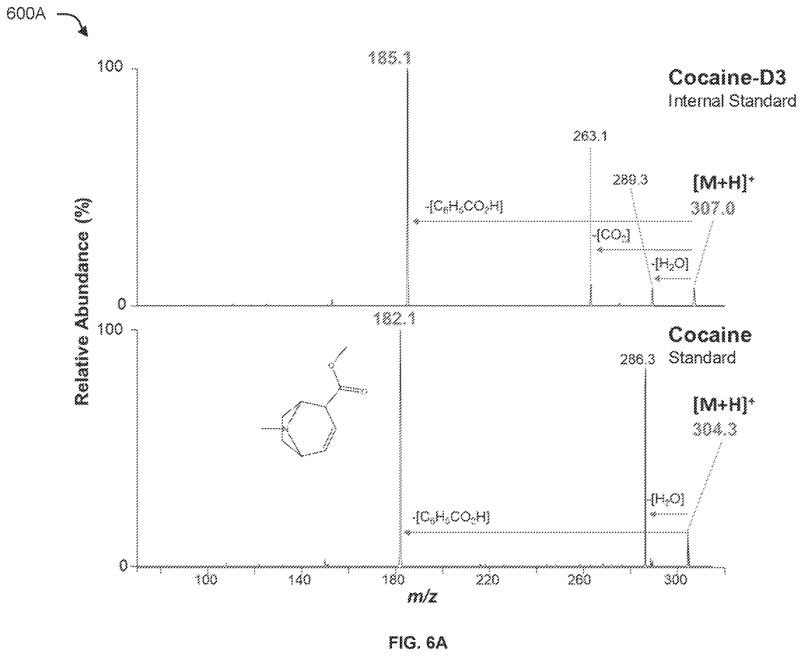

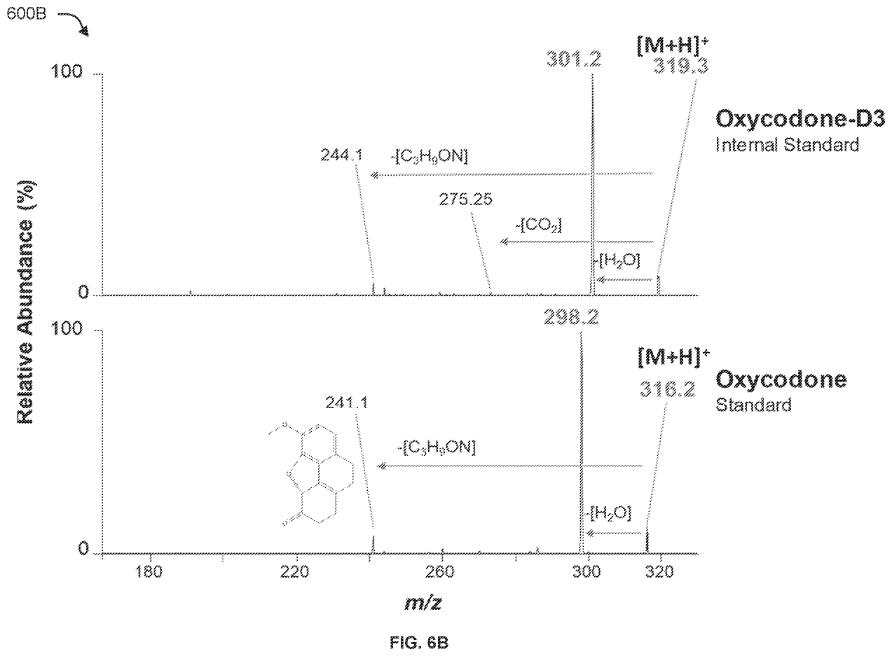

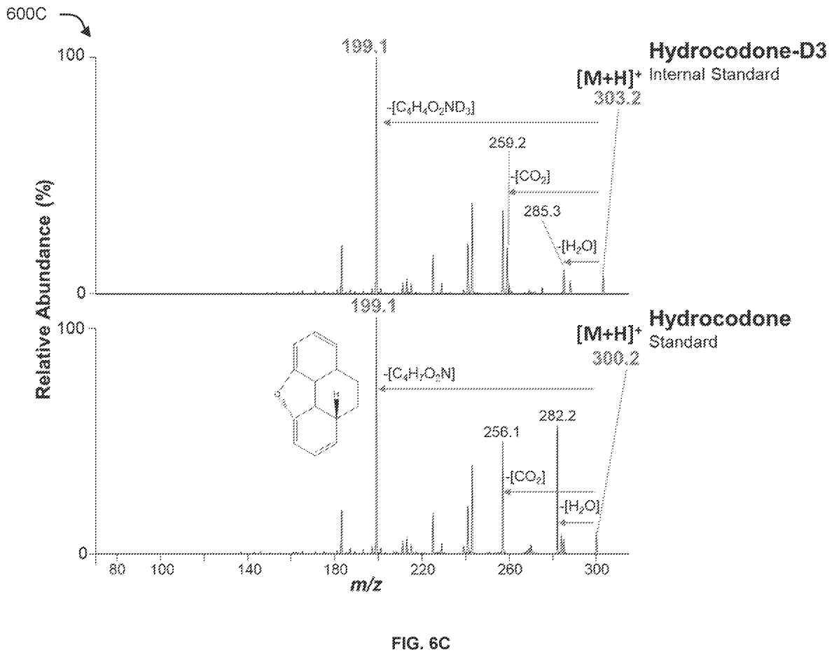

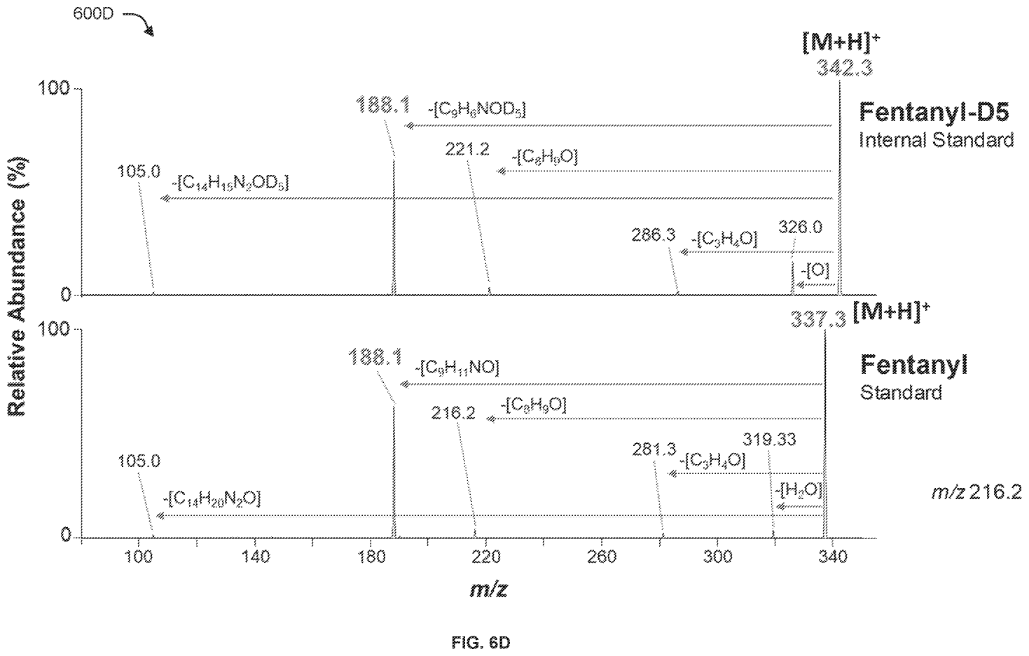

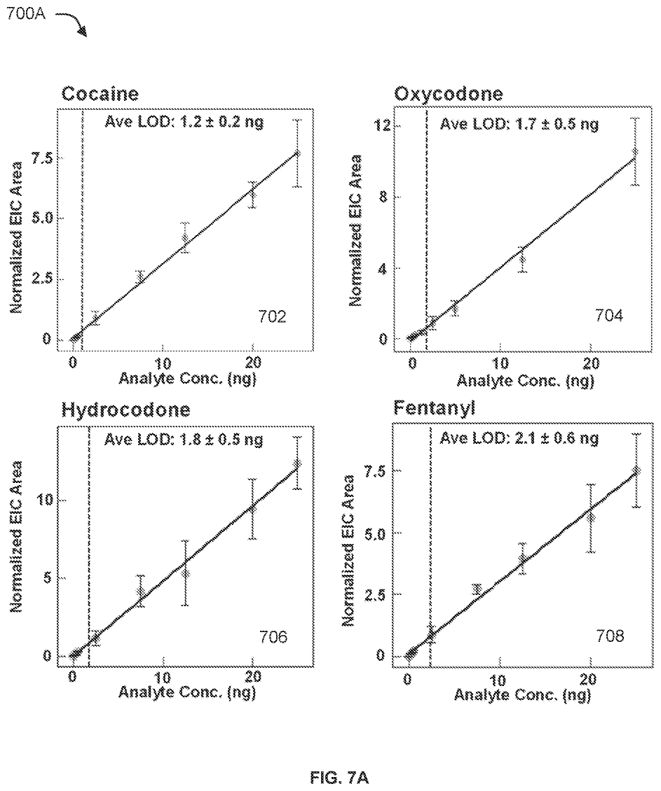

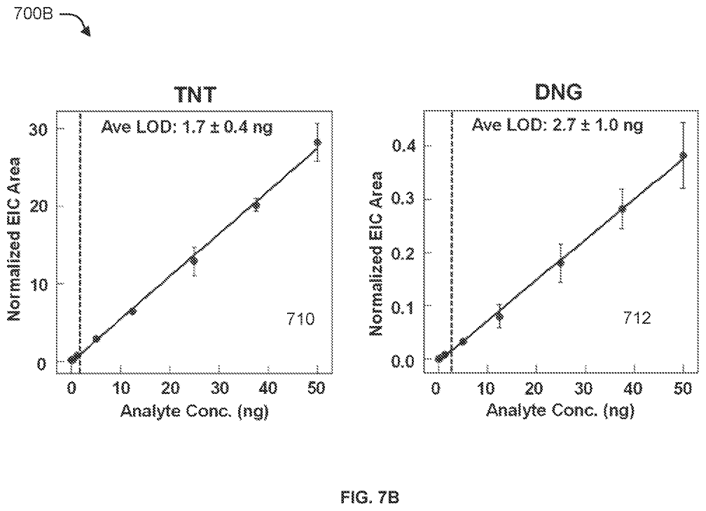

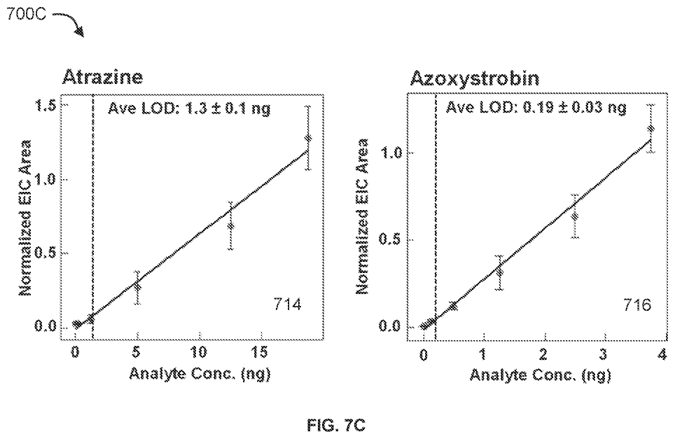

FIGS. 6A-6D are example mass spectra 600A, 600B, 600C, 600D of chemical standards including cocaine, oxycodone, hydrocodone, and fentanyl with and without deuterium labeling. In some implementations, the chemical standards may be measured using the chemical measurement system as shown in FIGS. 1-4 using the example process 500 as shown in FIG. 5 or in another manner. In some instances, the chemical standards without deuterium labeling on a sample surface and chemical standards with deuterium labeling in the liquid solvent (e.g., internal standards) are used for calibration purposes, e.g., develop calibration curves as shown in FIGS. 7A-7C. In some example, the chemical measurement system includes a Thermo Scientific Orbitrap XL hybrid mass spectrometer or another mass spectrometer.

In some implementations, four different locations on a sample surface may be analyzed and an analyte collected at each location is scanned ten times, yielding a total of forty analyses of a chemical compound at one concentration. In certain implementations, the process is repeated on at least three different concentrations. As shown in FIGS. 6A-6D, the mass spectra of deuterium labeled internal standards are similar to the mass spectra of respective standards without deuterium labeling. In some examples, the exact position (e.g., m/z ratio) may vary depending on the presence or absence of the deuterium labeling on the fragment ions. For example, when a deuterium labeling is retained in a particular fragment ion, the position of the peak corresponding to the fragment ion is shifted to a higher m/z value. Similarly, when a deuterium labeling is not retained on a particular fragment ion, the position of the peak corresponding to the fragment ion is shifted to a lower m/z value.

In some implementations, one or more particular fragment ions of interest that are representative of the chemical compounds may be selected for limit-of-detection (LOD) analysis. For example, the ion peaks at m/z=182.1 are used for determining the LOD for cocaine (FIG. 6A); the ion peaks at m/z=298.2 are used for determining the LOD for oxycodone (FIG. 6B); the ion peaks at m/z=199.1 are used for determining the LOD for hydrocodone (FIG. 6C); and the ion peaks at m/z=188.1 are used for determining the LOD for oxycodone (FIG. 6D).

FIGS. 7A-7C are plots 700A, 700B, 700C of calibration curves of chemical compounds. As shown in FIGS. 7A-7C, the chemical compounds that are measured include cocaine, oxycodone, hydrocodone, fentanyl, trinitrotoluene (TNT), dinitroglycerin (DNG),atrazine and azoxystrobin. In some implementations, a LOD is determined using three times the standard deviation and the slope of a fitted curve. In some instances, a sample surface is prepared by dropping a fixed volume of an analyte containing the chemical compound at a fixed concentration on the sample surface followed by drying in air for 30 mins. At least four replicates are deposited on the sample surface. Each of the at least four deposited replicates on the sample surface is collected and measured by a chemical measurement system, e.g., the chemical measurement system as shown in FIGS. 1-4.

As shown in 702, a first calibration curve for cocaine is determined based on the normalized ion intensity level at a m/z ratio of 182.1. The LOD as indicated by the dotted line is 1.2±0.2 ng, which is equivalent to 244±42 ppb. As shown in 704, a second calibration curve for oxycodone is determined based on the ion intensity level at a m/z ratio of 298. The LOD as indicated by the dotted line is 1.7±0.5 ng, which is equivalent to 332±98 ppb. As shown in 706, a third calibration curve for hydrocodone is determined based on the ion intensity level at a m/z ratio of 199.1. The LOD level as indicated by the dotted line is 1.8±0.5 ng, which is equivalent to 356±94 ppb. As shown in 708, a fourth calibration curve for fentanyl is determined based on the ion intensity level at a m/z ratio of 188.1. The LOD level as indicated by the dotted line is 2.1±0.6 ng, which is equivalent to 420±120 ppb. As shown in 710, a fifth calibration curve for TNT is determined based on the ion intensity level at a m/z ratio of 196. The LOD level as indicated by the dotted line is 1.7±0.4 ng, which is equivalent to 340±80 ppb. As shown in 712, a sixth calibration curve for DNG is determined based on the ion intensity level at a m/z ratio of 109. The LOD level as indicated by the dotted line is 2.7±1.0 ng, which is equivalent to 540±200 ppb. As shown in 714, a seventh calibration curve for atrazine is determined based on the ion intensity level at a m/z ratio of 174.0. The LOD level as indicated by the dotted line is 1.3±0.1 ng, which is equivalent to 254±28 ppb. As shown in 716, an eighth calibration curve for azoxystrobin is determined based on the intensity level at a m/z ratio of 404.1. The LOD level as indicated by the dotted line is 0.19±0.03 ng, which is equivalent to 38±6 ppb.

As shown in FIGS. 7A-7C, the LOD values for the controlled substances using the chemical measurement system and process as shown in FIGS. 1-5 are all below or within respective federal cutoff values established, for example, by the Department of Justice or the Environmental Protect Agency (EPA). In some implementations, the LOD values may be different or improved using another type of mass spectrometer, for example with triple quadrupoles.

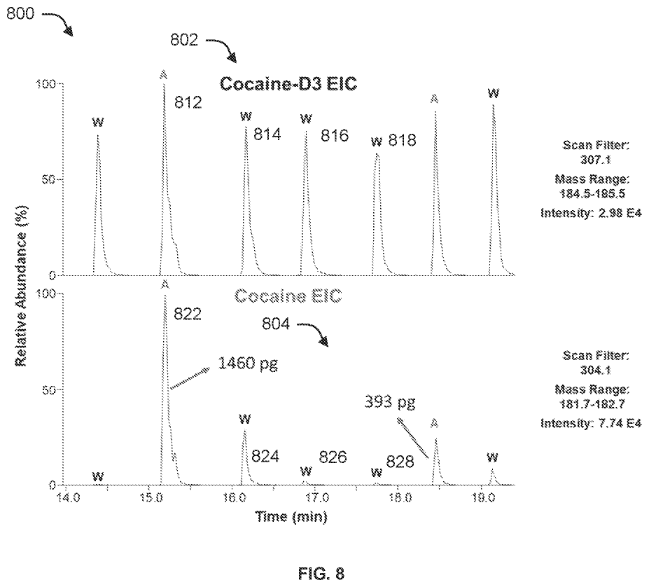

FIG. 8 are plots of extracted ion chromatograms 800 of signal intensities as a function of chromatographic retention time. As shown in FIG. 8, each of the EIC plots includes signal peaks at one or more selected m/z values in a series of recorded mass spectra. In some implementations, the series of mass spectra is collected from a surface of a dollar bill using the chemical measurement system as shown in FIGS. 1-4 and following the example process 500 as show in FIG. 5. In some instances, a liquid solvent used in the measurement contains a concentration of 25 ppb of deuterium labeled cocaine in a mixture of 99.9% of 1:1 methanol: water solution and 0.1% acidic acid. In some instances, a first portion of an analyte is extracted from an internal reservoir of a sampling probe after filling the internal reservoir for a first time period and maintaining the liquid solvent in the reservoir for a second time period. In some instances, the first time period is 2 seconds and the second time period is 3 seconds. In some instances, after the first portion of the analyte is extracted to the ionization system, the same area on the surface of the dollar bill is continuously flushed with the liquid solvent for three times generating second, third and fourth portions of the analyte. All four portions of the analyte are extracted to the ionization system and analyzed by the mass spectrometer of the chemical measurement system.

As shown in FIG. 8, a first panel 802 includes multiple peaks detected in a first m/z range of 184.5-185.5 from the series of mass spectra. In the first m/z range, only the signal peak at m/z=185.1 from the deuterium labeled cocaine can be detected as shown in FIG. 6A. Because the deuterium labeled cocaine is included in the liquid solvent delivered to the dollar bill surface in all the four portions of the analyte, peak intensity values of four consecutive peaks 812, 814, 816, 818 corresponding to the four portions remain consistent.

As shown in FIG. 8, a second panel 802 includes multiple peaks detected in a second m/z range of 181.7-182.7 from the series of mass spectra. In the second m/z range, only the signal peak at m/z=182.1 from the cocaine contained on the surface of the dollar bill can be detected as shown in FIG. 6A. Peak intensity values of four consecutive peaks 822, 824, 826, 828 corresponding to the four portions decrease with the increasing numbers of flushes using the liquid solvent. In some instances, the peak intensity value of the peak 822 corresponding to the first portion of the analyte may be compared to the calibration curve 702 as shown in FIG. 7 to determine the amount of cocaine on the dollar bill surface. For example, the amount of cocaine in the area measured by the sampling probe on the dollar bill surface is determined as 1460 pg.

Some of the subject matter and operations described in this specification can be implemented in digital electronic circuitry, or in computer software, firmware, or hardware, including the structures disclosed in this specification and their structural equivalents, or in combinations of one or more of them. Some of the subject matter described in this specification can be implemented as one or more computer programs, i.e., one or more modules of computer program instructions, encoded on a computer storage medium for execution by, or to control the operation of, data-processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media.

Some of the operations described in this specification can be implemented as operations performed by a data processing apparatus on data stored on one or more computer-readable storage devices or received from other sources.

The term “data-processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

In a general aspect of what is described above, chemical compounds are measured for forensic analysis.

In a first example, a chemical detection system includes a container, an ionization system, a mass spectrometer, one or more computer systems, a sampling probe, and a control system. The container includes a liquid solvent. The ionization system is configured to ionize an analyte. The mass spectrometer is configured to produce mass spectrometry data by processing the ionized analyte provided by the ionization system. The one or more computer systems are configured to analyze the mass spectrometry data to detect a chemical compound present on a sample surface. The sampling probe includes an internal reservoir, a first channel, a second channel, and a third channel. The internal reservoir is configured to hold a fixed volume of the liquid solvent in direct contact with the sample surface for a period of time to form the analyte in the sampling probe. The first channel is configured to communicate the liquid solvent into the internal reservoir. The second channel is configured to communicate gas into the internal reservoir. The third channel is configured to communicate the analyte from the internal reservoir. The control system is configured to perform operations including: supplying the liquid solvent to the internal reservoir through the first channel of the sampling probe; extracting the analyte from the internal reservoir through the third channel of the sampling probe and transferring the analyte to the ionization system by applying a first pressure in the ionization system, the first pressure being less than the atmospheric pressure in an environment of the chemical detection system.

Implementations of the first example may include one or more of the following features. The ionization system includes a transfer capillary configured to receive the analyte, and when the analyte is transferred, the analyte is transferred from the internal reservoir to the transfer capillary. The ionization system includes a heating element attached to the transfer capillary. When the analyte is ionized, the analyte is heated, by operation of the heating element, in the transfer capillary to obtain a vaporized analyte; and the vaporized analyte is ionized to produce the ionized analyte. The ionization system includes a chamber. When the analyte is transferred to the ionization system, the analyte is aspirated into the chamber of the ionization system through a tip of the transfer capillary.

Implementations of the first example may include one or more of the following features. The ionization system includes a discharge needle, and when the analyte is ionized, a discharge voltage is applied on the analyte using the discharge needle to obtain the ionized analyte. The discharge needle is positioned in proximity to a tip of the transfer capillary. The ionization system includes a chamber, the mass spectrometer includes an inlet tube residing in proximity to the tip of the transfer capillary and the discharge needle in the chamber. When the first pressure is applied, applying a vacuum on the inlet tube. The ionization system includes a chamber coupled to a vacuum source. When the first pressure is applied, the chamber is evacuated to the first pressure by operation of the vacuum source. The operations includes applying a second pressure on the inlet tube, wherein the second pressure on the inlet tube is less than the first pressure.

Implementations of the first example may include one or more of the following features. The system includes a first transfer tube that communicates the liquid solvent from the container to the first channel, and a second transfer tube that communicates the analyte from the sampling probe to the ionization system. The second channel includes an open end that receives air from the environment of the chemical detection system. The fixed volume is defined by the volume of the internal reservoir. The sampling probe is a handheld sampling probe. The sample surface includes a surface on a forensic object of interest. The sample surface includes a surface of paper money. The sample surface includes a surface of a biological tissue. When a chemical compound present on the sample surface is detected, a controlled substance on the sample surface is identified. When the controlled substance is identified, at least one of hydrocodone, cocaine, oxycodone, or fentanyl is identified. When the controlled substance is identified, an agrochemical substance on the sample surface is identified. When the controlled substance is identified, an explosive substance on the sample surface is identified.

In a second example, a liquid solvent is supplied through a first channel of a sampling probe to an internal reservoir of the sampling probe. A fixed volume of the liquid solvent in the internal reservoir is held in direct contact with a sample surface for a period of time to form an analyte in the sampling probe. Gas is supplied to the internal reservoir of the sampling probe through a second channel of the sampling probe. The analyte is extracted from the internal reservoir through a third channel of the sampling probe. The analyte is transferred to an ionization system by applying a first pressure in the ionization system, the first pressure being less than the atmospheric pressure in an environment of the sampling probe. By operation of the ionization system, the analyte from the internal reservoir is ionized. By operation of the mass spectrometer, mass spectrometry data is produced by processing the ionized analyte from the ionization system. The mass spectrometry data is analyzed to detect a chemical compound present at the sample surface.

Implementations of the second example may include one or more of the following features. The ionization system includes a transfer capillary that receives the analyte. When the analyte is transferred, the analyte is transferred from the sampling probe to the transfer capillary. The analyte is heated in the transfer capillary to obtain a vaporized analyte; and the vaporized analyte is ionized to produce the ionized analyte. The ionization system includes a chamber. When the analyte is transferred to the ionization system, the analyte is aspirated into the chamber of the ionization system through a tip of the transfer capillary.

Implementations of the second example may include one or more of the following features. The ionization system includes a discharge needle. When the analyte is ionized, a discharge voltage is applied on the analyte using the discharge needle to obtain the ionized analyte. The discharge needle is positioned in proximity to a tip of the transfer capillary. The ionization system includes a chamber. mass spectrometer includes an inlet tube residing in proximity to the tip of the transfer capillary and the discharge needle in the chamber. When the first pressure is applied, a vacuum is applied on the inlet tube. The ionized analyte is collected by the inlet tube and provided to the mass spectrometer. The ionization system includes a chamber coupled to a vacuum source. When the first pressure is applied, the chamber is evacuated to the first pressure by operation of the vacuum source. A second pressure is applied on the inlet tube, wherein the second pressure on the inlet tube is less than the first pressure.

Implementations of the second example may include one or more of the following features. The first channel receives the liquid solvent from an external container through a first transfer tube, and the analyte is transferred from the sampling probe to the mass spectrometer through a second transfer tube, and the second channel receives the gas through an open port that receives air from an atmosphere of the sampling probe. The fixed volume is defined by the volume of the internal reservoir. The sample surface includes a surface on a forensic object of interest. The sample surface includes a surface of paper money. The sample surface includes a surface of a biological tissue. When a chemical compound present on the sample surface is detected, a controlled substance on the sample surface is identified. When the controlled substance is identified, at least one of hydrocodone, cocaine, oxycodone, or fentanyl is identified. When the controlled substance is identified, an agrochemical substance on the sample surface is identified. When the controlled substance is identified, an explosive substance on the sample surface is identified.

While this specification contains many details, these should not be understood as limitations on the scope of what may be claimed, but rather as descriptions of features specific to particular examples. Certain features that are described in this specification or shown in the drawings in the context of separate implementations can also be combined. Conversely, various features that are described or shown in the context of a single implementation can also be implemented in multiple implementations separately or in any suitable sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

A number of embodiments have been described. Nevertheless, it will be understood that various modifications can be made. Accordingly, other embodiments are within the scope of the present disclosure.

1. A chemical detection system comprising:
a container comprising a liquid solvent;
an ionization system configured to ionize an analyte;
a mass spectrometer configured to produce mass spectrometry data by processing the ionized analyte provided by the ionization system;
one or more computer systems configured to analyze the mass spectrometry data to detect a chemical compound present on a sample surface; and
a sampling probe comprising:
an internal reservoir configured to hold a fixed volume of the liquid solvent in direct contact with the sample surface for a period of time to form the analyte in the sampling probe;
a first channel configured to communicate the liquid solvent into the internal reservoir;
a second channel configured to communicate gas into the internal reservoir; and
a third channel configured to communicate the analyte from the internal reservoir;
a control system configured to perform operations comprising:
supplying the liquid solvent to the internal reservoir through the first channel of the sampling probe;
extracting the analyte from the internal reservoir through the third channel of the sampling probe and
transferring the analyte to the ionization system by applying a first pressure in the ionization system, the first pressure being less than the atmospheric pressure in an environment of the chemical detection system;
wherein detecting the chemical compound present on the sample surface comprises identifying a controlled substance, an agrochemical substance, an explosive substance, or a combination thereof on the sample surface.

2. The system of claim 1, wherein:
the ionization system comprises a transfer capillary configured to receive the analyte, and
transferring the analyte comprises transferring the analyte from the internal reservoir to the transfer capillary.

3. (canceled)

4. (canceled)

5. The system of claim 2, wherein the ionization system comprises a discharge needle positioned in proximity to a tip of the transfer capillary, and ionizing the analyte comprises applying a discharge voltage on the analyte using the discharge needle to obtain the ionized analyte.

6. (canceled)

7. The system of claim 5, wherein the ionization system comprises a chamber, the mass spectrometer comprises an inlet tube residing in proximity to the tip of the transfer capillary and the discharge needle in the chamber, and applying the first pressure comprises applying a vacuum on the inlet tube.

8-10. (canceled)

11. The system of claim 1, wherein the second channel comprises an open end that receives air from an atmosphere of the sampling probe.

12. (canceled)

13. (canceled)

14. The system of claim 1, wherein the sample surface comprises a surface on a forensic object of interest.

15. (canceled)

16. (canceled)

17. The system of claim 1, wherein detecting the chemical compound present on the sample surface comprises identifying a controlled substance on the sample surface, and identifying the controlled substance comprises identifying at least one of hydrocodone, cocaine, oxycodone, or fentanyl.

18-20. (canceled)

21. A method comprising:
supplying a liquid solvent through a first channel of a sampling probe to an internal reservoir of the sampling probe;
holding a fixed volume of the liquid solvent in the internal reservoir in direct contact with a sample surface for a period of time to form an analyte in the sampling probe;
supplying gas to the internal reservoir of the sampling probe through a second channel of the sampling probe;
extracting the analyte from the internal reservoir through a third channel of the sampling probe and transferring the analyte to an ionization system by applying a first pressure in the ionization system, the first pressure being less than the atmospheric pressure in an environment of the sampling probe;
ionizing, by operation of the ionization system, the analyte from the internal reservoir;
by operation of the mass spectrometer, producing mass spectrometry data by processing the ionized analyte from the ionization system; and
analyzing the mass spectrometry data to detect a chemical compound present at the sample surface;
wherein detecting the chemical compound present on the sample surface comprises identifying a controlled substance, an agrochemical substance, an explosive substance, or a combination thereof at the sample surface.

22. The method of claim 21, wherein:
the ionization system comprises a transfer capillary that receives the analyte, and
transferring the analyte comprises transferring the analyte from the sampling probe to the transfer capillary.

23. The method of claim 22, wherein ionizing the analyte comprises:
heating the analyte in the transfer capillary to obtain a vaporized analyte; and
ionizing the vaporized analyte to produce the ionized analyte.

24. The method of claim 22, wherein the ionization system comprises a chamber, and transferring the analyte to the ionization system comprises aspirating the analyte into the chamber of the ionization system through a tip of the transfer capillary.

25. The method of claim 22, wherein the ionization system comprises a discharge needle positioned in proximity to a tip of the transfer capillary, and ionizing the analyte comprises applying a discharge voltage on the analyte using the discharge needle to obtain the ionized analyte.

26. (canceled)

27. The method of claim 25, wherein the ionization system comprises a chamber, the mass spectrometer comprises an inlet tube residing in proximity to the tip of the transfer capillary and the discharge needle in the chamber, and applying the first pressure comprises applying a vacuum on the inlet tube.

28. The method of claim 27, wherein the ionized analyte is collected by the inlet tube and provided to the mass spectrometer.

29. The method of claim 27, comprising applying a second pressure on the inlet tube, wherein the second pressure on the inlet tube is less than the first pressure.

30. The method of claim 21, wherein the ionization system comprises a chamber coupled to a vacuum source, and applying the first pressure comprises evacuating the chamber to the first pressure by operation of the vacuum source.

31. (canceled)

32. (canceled)

33. The method of claim 21, wherein the sample surface comprises a surface on a forensic object of interest.

34. The method of claim 21, wherein the sample surface comprises a surface of paper money.

35. The method of claim 21, wherein the sample surface comprises a surface of a biological tissue.

36. The method of claim 21, wherein detecting a chemical compound present on the sample surface comprises identifying a controlled substance on the sample surface, and identifying the controlled substance comprises identifying at least one of hydrocodone, cocaine, oxycodone, or fentanyl.

37-39. (canceled)
